(19) United States
(12) Patent Application Publication
(10) Pub. No.: US 2026/0283850 A1
(43) Pub. Date: Sep. 24, 2026

(54) INTRAOCULAR LENS WITH INCREASED DEPTH OF FOCUS AND METHODS FOR INCREASING A DEPTH OF FOCUS OF AN INTRAOCULAR LENS

(71) Applicant: Alcon Inc., Fribourg (CH)

(72) Inventors: Zsolt BOR, Lake Forest, CA (US); Raul MAURICIO, Mission Viejo, CA (US)

(73) Assignee: Alcon Inc., Fribourg (CH)

(21) Appl. No.: 19/449,303

(22) Filed: Jan. 14, 2026

Related U.S. Application Data
(60) Provisional application No. 63/775,543, filed on Mar. 21, 2025.

(51) Int. Cl. A61F 9/008; A61F 2/16

(52) U.S. Cl. CPC A61F 9/00834; A61F 2/16; A61F 2002/1681; A61F 2009/0087

(57) ABSTRACT

Disclosed are intraocular lenses with increased depth of focus and methods for increasing a depth of focus of an intraocular lens (IOL). In some embodiments, a method of increasing a depth of focus of an IOL can comprise determining a location of a visual axis of an eye comprising an IOL implanted therein, adjusting a refractive power of the eye with the IOL implanted therein to between about −0.7 diopters and about −0.9 diopters, and directing laser light at the optic portion in a spiral pattern until an opaque mask is formed within part of the optic portion enclosing a pinhole aligned with the visual axis.

### CROSS-REFERENCE TO RELATED APPLICATION

This application claims the benefit of priority to U.S. Provisional Patent Application No. 63/775,543 filed on Mar. 21, 2025, the content of which is incorporated herein by reference in its entirety.

### TECHNICAL FIELD

The present disclosure relates generally to the field of intraocular lenses, and, more specifically, to intraocular lenses with increased depth of focus and methods for increasing a depth of focus of an intraocular lens.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the crystalline lens matrix from the patient's lens capsule and replaces it with an intraocular lens (IOL).

However, there may be a need to adjust such IOLs post-operatively or after implantation within the eye of a patient. For example, once an IOL is implanted within the capsular bag, an aggressive healing response by tissue within the capsular bag can squeeze the IOL and change the optical power. Moreover, a patient's cornea or muscles within the eye may change as a result of injury, disease, or aging. In such cases, it may be necessary to adjust the patient's implanted IOL to account for such changes. For example, it may be necessary to increase the depth of focus of the patient's implanted IOL to enhance visual acuity at near or intermediate distances.

Therefore, a solution is needed which allows for post-implant adjustment of IOLs to correct certain residual refractive errors without having to undergo additional invasive surgery. Such a solution should not be overly complicated and still allow such IOLs to be cost-effectively made.

### SUMMARY

Disclosed herein are intraocular lenses with increased depth of focus and methods for increasing a depth of focus of an intraocular lens (IOL). In some embodiments, a method of increasing a depth of focus of an IOL can comprise determining a location of a visual axis of an eye comprising an IOL implanted therein, adjusting a refractive power of the eye with the IOL implanted therein to between about −0.7 diopters and about −0.9 diopters, and directing laser light at the optic portion in a spiral pattern until an opaque mask is formed within part of the optic portion enclosing a pinhole. The pinhole can be made to align or be centered with the visual axis.

In some embodiments, the pinhole can be shaped substantially as a circle.

In some embodiments, the pinhole can be shaped substantially as an ellipse.

In some embodiments, the pinhole can be a non-opaque or transparent portion of the optic portion surrounded by the opaque mask.

In some embodiments, the pinhole can have a pinhole diameter. For example, the pinhole diameter can be between about 1.0 mm and about 3.0 mm. As a more specific example, the pinhole diameter can be between about 1.8 mm and about 2.2 mm.

In some embodiments, the opaque mask can have a mask outer diameter or mask width. For example, the mask outer diameter or the mask width can be between about 3.0 mm and about 5.0 mm.

In some embodiments, the laser light directed at the optic portion can be generated by a femtosecond laser.

In some embodiments, the laser light can have a wavelength of between about 1030 nm and about 1064 nm.

In some embodiments, the opaque mask can be a black-colored mask.

In some embodiments, the opaque mask can be shaped substantially as an annulus.

In some embodiments, the opaque mask can be shaped substantially as an elliptical ring.

In some embodiments, the opaque mask can be comprised of a plurality of opaque spots densely packed together within the optic portion. Each of the opaque spots can have a spot diameter. For example, the spot diameter can be between about 3 μm and about 4 μm.

In some embodiments, the opaque mask can be an apodized transmission mask such that a density of the opaque spots is greater at a peripheral edge of the opaque mask than a portion of the opaque mask radially inward of the peripheral edge.

In some embodiments, the method can further comprise implanting the IOL within the eye of a subject and waiting for at least 90 days or three months after the IOL is implanted before determining the location of the visual axis.

In some embodiments, the pinhole is made to align with the visual axis when at least part of the pinhole overlaps with the visual axis.

In some embodiments, the opaque mask can comprise multiple mask layers. At least one of the mask layers can be unaligned or staggered with respect to another one of the mask layers in an anteroposterior direction.

In some embodiments, each of the mask layers can be spaced apart from one another in the anteroposterior direction.

In some embodiments, the optic portion can be made of a biocompatible polymeric material and directing the laser light at the optic portion can further comprise directing the laser light at a portion of the biocompatible material until the portion of the biocompatible material turns opaque.

Also disclosed is another embodiment of a method of forming a pinhole intraocular lens (IOL). The method can comprise directing laser light at a first layer of an optic portion of an IOL in a first spiral pattern until a first opaque mask layer is formed within the optic portion. The IOL can have a refractive power configured to adjust a refractive power of an eye to between about −0.7 diopters and about −0.9 diopters when the IOL is implanted within the eye. The method can also comprise directing the laser light at a second layer of the optic portion of the IOL in a second spiral pattern until a second opaque mask layer is formed within the optic portion. The first opaque mask layer can be unaligned or staggered with respect to the second opaque mask layer in an anteroposterior direction.

In some embodiments, the method can further comprise directing the laser light at a third layer of the optic portion of the IOL in a third spiral pattern until a third opaque mask layer is formed within the optic portion. The second opaque mask layer can be unaligned or staggered with respect the third opaque mask layer in an anteroposterior direction.

In some embodiments, the first opaque mask layer and the second opaque mask layer can define a pinhole. In additional embodiments, the third opaque mask layer can also define a pinhole.

In some embodiments, the pinhole can be shaped substantially as a circle.

In some embodiments, the pinhole can be shaped substantially as an ellipse.

In some embodiments, the pinhole can be a non-opaque or transparent portion of the optic portion.

In some embodiments, the pinhole can have a pinhole diameter. For example, the pinhole diameter can be between about 1.0 mm and about 3.0 mm. As a more specific example, the pinhole diameter can be between about 1.8 mm and about 2.2 mm.

In some embodiments, the method can further comprise determining a location of a visual axis of an eye when the IOL is implanted in the eye. The pinhole can be made to align or be centered with the visual axis.

In some embodiments, the pinhole can be made to align with the visual axis when at least part of the pinhole overlaps with the visual axis.

In some embodiments, the laser light can be generated by a femtosecond laser.

In some embodiments, the laser light can have a wavelength of between about 1030 nm and about 1064 nm.

In some embodiments, at least one of the first opaque mask layer and the second opaque mask layer can be a black-colored mask layer.

In some embodiments, at least one of the first opaque mask layer and the second opaque mask layer can be shaped substantially as an annulus.

In some embodiments, at least one of the first opaque mask layer and the second opaque mask layer can be shaped substantially as an elliptical ring.

In some embodiments, at least one of the first opaque mask layer and the second opaque mask layer can be comprised of a plurality of opaque spots densely packed together within the optic portion. Each of the opaque spots can have a spot diameter. For example, the spot diameter can be between about 3 μm and about 4 μm.

In some embodiments, at least one of the first opaque mask layer and the second opaque mask layer can be an apodized transmission mask layer such that a density of the opaque spots is greater at a peripheral edge than a portion radially inward of the peripheral edge.

In some embodiments, the first opaque mask layer can be spaced apart from the second opaque mask layer in the anteroposterior direction.

In some embodiments, the optic portion can be made of a biocompatible polymeric material. In these embodiments, the step of directing the laser light at the first layer and the second layer of the optic portion can further comprise directing the laser light at layers of the biocompatible material making up the optic portion until the layers of the biocompatible material turn opaque.

Further disclosed is an intraocular lens (IOL) comprising an optic portion and one or more haptics coupled to the optic portion. The optic portion can comprise an opaque mask formed within the optic portion. The opaque mask can enclose a pinhole. The opaque mask can comprise multiple mask layers. At least one of the multiple mask layers can be unaligned or staggered with respect to another one of the multiple mask layers in an anteroposterior direction.

In some embodiments, the optic portion of the IOL can have a refractive power configured to adjust a refractive power of an eye to between about −0.7 diopters and about −0.9 diopters when the IOL is implanted within the eye.

In some embodiments, the pinhole can be aligned or centered with a visual axis of the eye when the IOL is implanted within the eye.

In some embodiments, the pinhole can be aligned with the visual axis when at least part of the pinhole overlaps with the visual axis.

In some embodiments, the pinhole can be shaped substantially as a circle.

In some embodiments, the pinhole can be shaped substantially as an ellipse.

In some embodiments, the pinhole can be a non-opaque portion of the optic portion surrounded by the opaque mask.

In some embodiments, the pinhole can have a pinhole diameter. For example, the pinhole diameter can be between about 1.0 mm and about 3.0 mm. As a more specific example, the pinhole diameter can be between about 1.8 mm and about 2.2 mm.

In some embodiments, the opaque mask can have a mask outer diameter or mask width. For example, the mask outer diameter or the mask width can be between about 3.0 mm and about 5.0 mm.

In some embodiments, each of the mask layers can be spaced apart from one another in the anteroposterior direction.

In some embodiments, the optic portion can be made of a biocompatible polymeric material. The opaque mask can be made by directing laser light at a portion of the biocompatible material making up the optic portion until the portion of the biocompatible material turns opaque.

In some embodiments, the laser light can be generated by a femtosecond laser.

In some embodiments, the laser light can have a wavelength of between about 1030 nm and about 1064 nm.

In some embodiments, the opaque mask can be a black-colored mask.

In some embodiments, the opaque mask can be shaped substantially as an annulus.

In some embodiments, the opaque mask can be shaped substantially as an elliptical ring.

In some embodiments, the opaque mask can be comprised of a plurality of opaque spots densely packed together within the optic portion.

In some embodiments, each of the opaque spots can have a spot diameter. The spot diameter can be between about 3 μm and about 4 μm.

In some embodiments, the opaque mask can be an apodized transmission mask such that a density of the opaque spots is greater at a peripheral edge of the opaque mask than a portion of the opaque mask radially inward of the peripheral edge.

### BRIEF DESCRIPTION OF THE DRAWINGS

FIG. 1 illustrates a top plan view of an embodiment of an intraocular lens comprising an opaque mask defining a pinhole aperture configured to increase a depth of focus of the intraocular lens.

FIG. 2A illustrates one embodiment of a method of increasing a depth of focus of an intraocular lens.

FIG. 2B illustrates one embodiment of a method of determining a location of a visual axis of an eye comprising an intraocular lens implanted therein.

FIG. 3A illustrates an embodiment of the opaque mask of the intraocular lens with an elliptical-shaped pinhole.

FIG. 3B illustrates an embodiment of an elliptical-shaped opaque mask with an elliptical-shaped pinhole.

FIG. 4 illustrates that the opaque mask can be comprised of multiple mask layers and at least one of the mask layers can be unaligned with another one of the mask layers in an anteroposterior direction.

FIG. 5 illustrates that an annular-shaped opaque mask can be created by directing laser light at the optic portion in a spiral pattern.

FIG. 6 is a close-up image of opaque dots making up part of an opaque mask.

FIG. 7 is an image of one embodiment of an intraocular lens comprising a substantially annular-shaped opaque mask defining a pinhole aperture.

### DETAILED DESCRIPTION

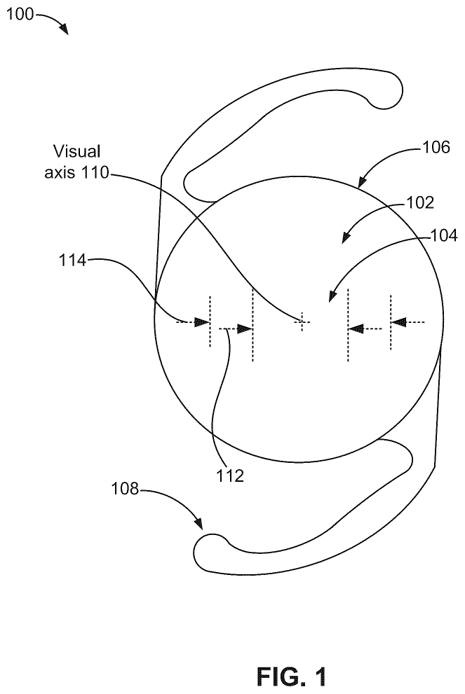

FIG. 1 illustrates a top plan view of an embodiment of an intraocular lens 100 comprising an opaque mask 102 defining a pinhole 104 or aperture. The pinhole 104 can be configured to increase a depth of focus of the intraocular lens 100.

The intraocular lens 100 can be implanted within an eye of a subject to correct for defocus aberration, corneal astigmatism, spherical aberration, or a combination thereof. For example, the intraocular lens 100 can be positioned within a native capsular bag in which a native lens has been removed.

In some embodiments, the opaque mask 102 can be formed within an optic portion 106 of the intraocular lens 100. In certain embodiments, the intraocular lens 100 can comprise the optic portion 106 and one or more haptics 108 coupled to and extending peripherally from the optic portion 106. For example, as shown in FIG. 1, the intraocular lens 100 can comprise two haptics 108. As a more specific example, the two haptics 108 can be positioned substantially diametrically opposed to one another. When the intraocular lens 100 is implanted within the native capsular bag, the optic portion 106 of the intraocular lens 100 can be configured to refract light that enters the eye onto the retina.

In certain embodiments, the intraocular lens 100 can be a one-piece lens such that the haptics 108 are connected to and extend from the optic portion 106. In this example embodiment, the haptics 108 are formed along with the optic portion 106 and are not adhered or otherwise coupled to the optic portion 106 in a subsequent step. In other embodiments, the haptics 108 can be coupled to and adhered to the optic portion 106. For example, the haptics 108 can be adhered to the optic portion 106 after each is formed separately.

In some embodiments, the optic portion 106 can have an optic portion diameter. The optic portion diameter can be between about 5.0 mm and 8.0 mm. For example, the optic portion diameter can be about 6.0 mm.

In some embodiments, the intraocular lens 100 can have a total device length as measured from a haptic distal end of a first haptic to the haptic distal end of a second haptic (in instances where the intraocular lens 100 has two haptics 108). For example, the total device length can be between about 10.0 mm and about 15.0 mm. As a more specific example, the total device length can be between about 11.0 mm and about 13.0 mm.

In some embodiments, the opaque mask 102 can enclose, surround, or substantially encircle the pinhole 104. The pinhole 104 can be a non-opaque or transparent portion of the optic portion 106 enclosed or surrounded by the opaque mask 102.

As shown in FIG. 1, the pinhole 104 or aperture can be aligned or made to align with a visual axis 110 of the eye of a subject when the intraocular lens 100 is implanted within the eye.

In some embodiments, the pinhole 104 can be shaped substantially as a circle (when the intraocular lens 100 is viewed in an en face or top-down direction). In these embodiments, the opaque mask 102 can be substantially shaped as an annulus.

In other embodiments, the pinhole 104 can be shaped substantially as an ellipse (when the intraocular lens 100 is viewed in an en face or top-down direction).

In certain embodiments, the pinhole 104 can have a pinhole diameter 112 or pinhole width. For example, the pinhole diameter 112 or the pinhole width can be between about 1.0 mm and about 3.0 mm. As a more specific example, the pinhole diameter 112 or the pinhole width can be between about 1.8 mm and about 2.2 mm.

The opaque mask 102 can have a mask outer diameter 114 or mask width. In some embodiments, the mask outer diameter 114 or the mask width can be between about 3.0 mm and 5.0 mm.

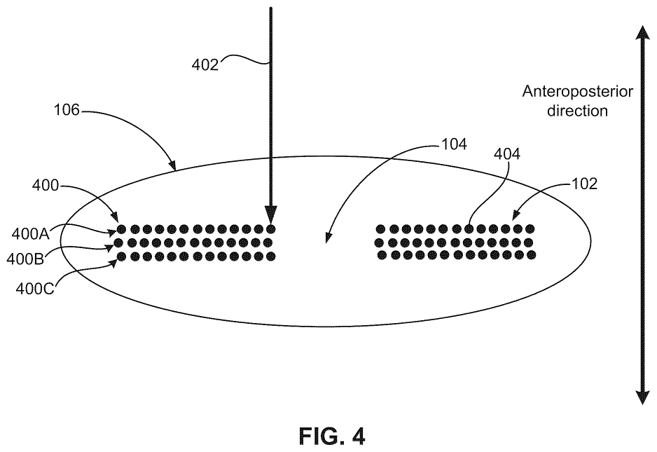

In some embodiments, the opaque mask 102 can comprise multiple mask layers (see, also, FIG. 4). As will be discussed in more detail in later sections, at least one of the multiple mask layers can be unaligned or staggered with respect to another one of the multiple mask layers in an anteroposterior direction.

In some embodiments, the intraocular lens 100 can be made at least in part of a hydrophobic acrylic material. For example, the optic portion 106 can be made at least in part of the hydrophobic acrylic material. In certain embodiments, both the optic portion 106 and the haptics 108 can be made of the same hydrophobic acrylic material. As a more specific example, the intraocular lens 100 can be made at least in part of a hydrophobic acrylate/methacrylate copolymer.

In some embodiments, the intraocular lens 100 can be made at least in part of a hydrophobic acrylic material comprising hydroxyethyl methacrylate (HEMA) and/or hydroxyethyl acrylate (HEA). In some embodiments, the intraocular lens 100 can be made at least in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably, unless otherwise specified. For example, the cross-linked copolymer can comprise an alkyl acrylate or methacrylate, a fluoro-alkyl acrylate or fluoro-alkyl methacrylate, and a phenyl-alkyl acrylate. In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate. The final composition of the cross-linked copolymer can also comprise a cross-linker or cross-linking agent, such as ethylene glycol dimethacrylate (EGDMA), a hydroxyl-functional acrylic monomer (e.g., HEMA or HEA), certain initiators or initiating agents, and a UV absorber.

In some embodiments, the optic portion 106 can be designed to have a refractive power that can adjust a refractive power of the eye of the subject to between about −0.7 diopters and about −0.9 diopters when the intraocular lens 100 is implanted within the eye.

In some embodiments, the intraocular lens 100 can be a negative power lens when the subject is emmetropic or is moderately or severely farsighted or hyperopic.

In other embodiments, the intraocular lens 100 can be a positive power lens when the subject is moderately or severely nearsighted or myopic.

In some embodiments, neither the optic portion 106 nor the haptic(s) 108 comprise a chamber or lumen filled with a fluid.

In alternative embodiments, the optic portion 106 can be filled with a fluid. For example, the optic portion 106 can comprise an anterior element, a posterior element, and an optic fluid chamber defined therebetween. The optic fluid chamber can be filled with a fluid. In these embodiments, at least one of the haptics 108 can comprise a haptic fluid lumen extending through at least part of the haptic 108. The haptic fluid lumen(s) can also be filled with the fluid and can be in fluid communication with the optic fluid chamber. In certain embodiments, a base power of the optic portion 106 can be configured to change based on an internal fluid pressure within the fluid-filled optic fluid chamber. The base power of the optic portion 106 can be configured to increase or decrease as fluid enters or exits the fluid-filled optic fluid chamber. For example, the base power of the optic portion 106 can be configured to decrease as fluid exits or is drawn out of the fluid-filled optic fluid chamber into the haptic fluid lumen(s). Also, for example, the base power of the optic portion 106 can be configured to increase as fluid enters the fluid-filled optic fluid chamber from the haptic fluid lumen(s).

In some embodiments, the fluid within the optic fluid chamber and the haptic fluid lumen(s) can be an oil. More specifically, in certain embodiments, the fluid within the optic fluid chamber and the haptic fluid lumen(s) can be a silicone oil or fluid. For example, the fluid can be a silicone polymer containing aliphatic or aromatic groups, or combinations thereof.

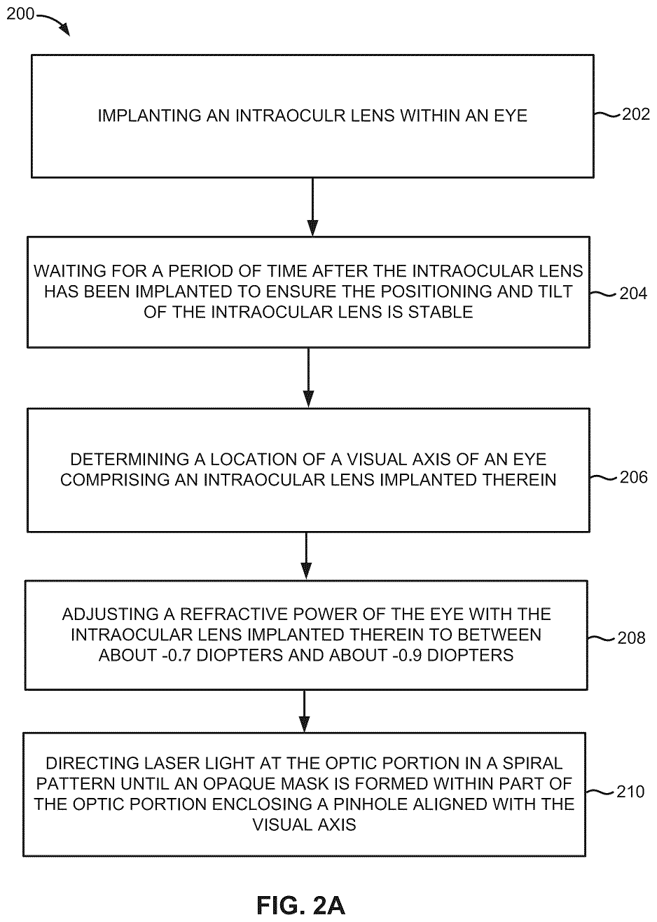

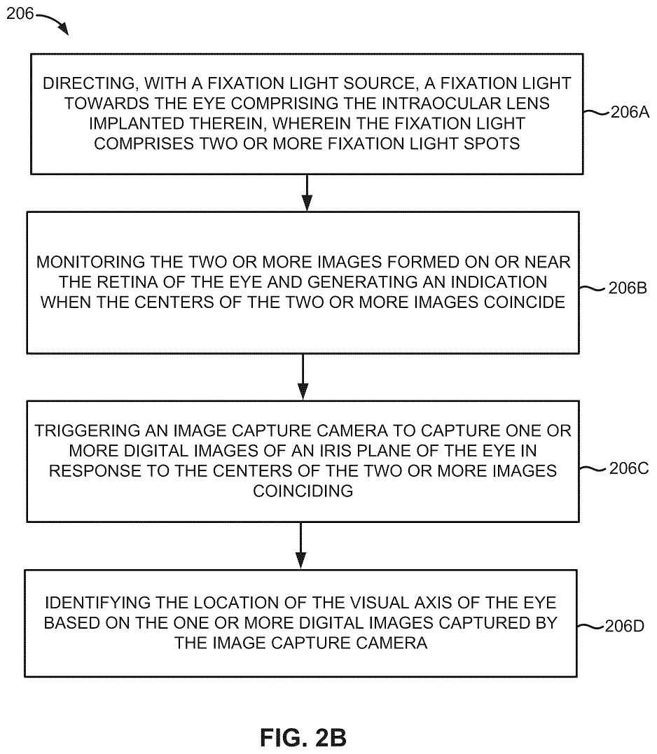

FIG. 2A illustrates one embodiment of a method 200 of increasing a depth of focus of an intraocular lens. The method 200 can comprise implanting an intraocular lens within an eye of a subject in operation 202. For example, the implantation procedure can involve introducing the intraocular lens into a capsular bag of the subject after the native crystalline lens has been removed as part of a phacoemulsification procedure. As a more specific example, the intraocular lens can be introduced after the native crystalline lens is emulsified using an ultrasonic phacoemulsification probe and aspirated from the capsular bag.

In some embodiments, the intraocular lens can be a monofocal IOL. For example, the intraocular lens can be the Clareon® monofocal IOL or the AcrySof® monofocal IOL.

In other embodiments, the intraocular lens can be an adjustable monofocal IOL.

In alternative embodiments, the intraocular lens can be a multifocal IOL.

In additional embodiments, the intraocular lens can be a fluid-filled IOL such as an accommodating IOL (or “AIOL”). Examples of AIOLs are discussed in the following U.S. patent publications: U.S. Pat. Pub. No. 2018/0153682 and in the following issued U.S. patents: U.S. Pat. Nos. 11,744,697; 11,660,182; 11,622,850; 11,426,270; 10,433,949; 10,299,913; 10,195,020; and 8,968,396, the contents of which are incorporated herein by reference in their entireties.

As will be discussed in more detail in later sections, the intraocular lens can also be a fluid-tunable non-accommodating intraocular lens. Examples of fluid-tunable non-accommodating IOLs or non-accommodating static-focus adjustable IOLs are discussed in U.S. Pat. No. 11,471,272, the content of which is incorporated herein by reference in its entirety.

The method 200 can also comprise waiting for a period of time after the intraocular lens has been implanted within the eye to ensure that the healing process is complete and the x/y/z position and tilt of the implanted intraocular lens is stabilized and not changing in operation 204. In some embodiments, the period of time can be between about 90 days and about 120 days. In other embodiments, the period of time can be between about 70 days and about 90 days.

The method 200 can also comprise determining a location of a visual axis of the eye comprising the intraocular lens implanted therein in operation 206. One example procedure for determining the location of the visual axis of the eye is discussed in relation to FIG. 2B.

In alternative embodiments, the visual axis of the eye can be approximated to be halfway between the pupil center and the corneal vertex. As previously discussed, the method 200 can comprise waiting between about 70 days and 120 days after the intraocular lens is implanted before determining the location of the visual axis.

The method 200 can also comprise adjusting a refractive power of the eye with the intraocular lens implanted therein to between about −0.7 diopters and about −0.9 diopters in operation 208. In some embodiments, adjusting the refractive power of the eye with the intraocular lens implanted therein to between about −0.7 diopters and about −0.9 diopters can further comprise using lasers to modify the anterior curvature of the cornea in such a way that the refractive power of the eye is changed. For example, the refractive power of the eye can be adjusted using laser procedures such as photorefractive keratectomy (PRK) or laser-assisted in situ keratomileusis (LASIK).

One unexpected discovery made by the applicant is that the outcome of the method 200 disclosed herein is improved when the refractive power of the eye with the intraocular lens implanted therein is adjusted to between about −0.7 diopters and about −0.9 diopters.

In some embodiments, operation 208 is not necessary if the refractive power of the eye with the intraocular lens implanted therein is already between about −0.7 diopters and about −0.9 diopters.

The method 200 can further comprise directing laser light at the optic portion of the intraocular lens implanted in the eye in a spiral pattern until an opaque mask is formed within part of the optic portion enclosing a pinhole aligned with the visual axis in operation 210. The pinhole is aligned with the visual axis when at least part of the pinhole overlaps with the visual axis. Directing laser light at the optic portion of the intraocular lens will be discussed in more detail in relation to FIG. 4.

The laser light directed at the optic portion of the intraocular lens implanted within the eye can be generated by a femtosecond laser. For example, the laser light can have a wavelength of between about 1030 nm and 1064 nm (e.g., about 1053 nm).

In some embodiments, the end result of method 200 is to transform an implanted intraocular lens (e.g., an implanted monofocal IOL) into an extended depth of focus intraocular lens (see, e.g., the intraocular lens 100 of FIG. 1). For example, the depth of focus of an implanted intraocular lens can be increased up to about 3 diopters using the method 200 disclosed herein.

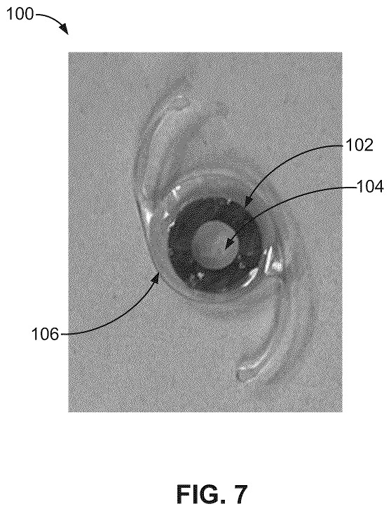

In some embodiments, the intraocular lens produced by the method 200 can be the intraocular lens described with respect to FIG. 1 and FIG. 7.

FIG. 2B illustrates certain suboperations or sub-steps of operation 206 for determining a location of a visual axis of an eye comprising an intraocular lens implanted therein. As previously discussed, the operation 206 can be considered part of a method 200 for increasing the depth of focus of an intraocular lens (see FIG. 2A).

The operation 206 can comprise directing, with a fixation light source, a fixation light towards the eye of the subject in operation 206A. The eye of the subject is the eye having the intraocular lens implanted therein from operation 202 (see FIG. 2A). In some embodiments, the fixation light can comprise two or more fixation light spots formed at different positions along an optical axis of the fixation light source. The fixation light spots can correspond with two or more images formed on or near a retina of the eye.

The operation 206 can also comprise monitoring, using a retina monitoring camera focused on the retina of the eye, the two or more images formed on or near the retina of the eye and generating an indication (e.g., an alert, a signal, a triggering command, etc.) when the centers of the two or more images coincide in operation 206B. The operation 206 can further comprise triggering an image capture camera to capture one or more digital images of an iris plane of the eye in response to the centers of the two or more images coinciding in operation 206C. The optical axis of the image capture camera is aligned with the optical axis of the fixation light source. In some embodiments, the optical axis is marked in the visual field of the image capture camera.

The operation 206 can further comprise identifying the location of the visual axis of the eye based on the one or more digital images captured by the image capture camera in operation 206D. For example, the visual axis of the eye can correspond with an X/Y location of the optical axis of the image capture camera relative to an X/Y location of the eye displayed in the one or more digital images. Also, for example, the one or more digital images can be analyzed to determine an average X/Y location of the optical axis of the image capture camera. In this example embodiment, the visual axis of the eye corresponds to the average X/Y location of the optical axis of the image capture camera.

In addition to the method steps disclosed herein, operation 206 can also comprise one or more steps disclosed in U.S. Pat. Pub. No. 2021/0369105, the content of which is incorporated herein by reference in its entirety.

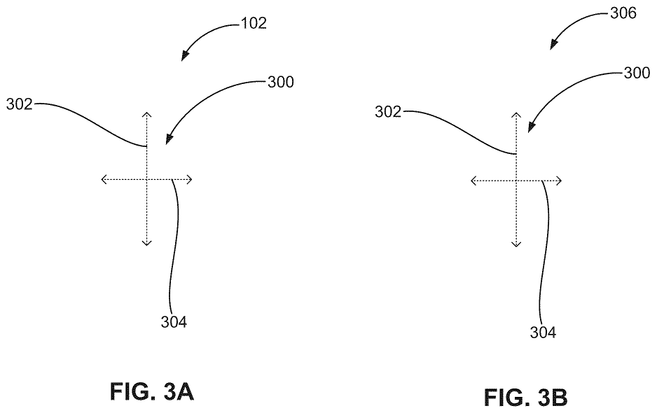

FIG. 3A illustrates an embodiment of the opaque mask 102 of the intraocular lens 100 with an elliptical-shaped pinhole 300. The elliptical-shaped pinhole 300 can be a non-opaque or transparent portion of the optic portion 106 of the intraocular lens 100 surrounded by the opaque mask 102.

As shown in FIG. 3A, the elliptical-shaped pinhole 300 can have a major axis 302 and a minor axis 304. The length of the major axis 302 can be greater than the length of the minor axis 304.

In some embodiments, the length of the minor axis 304 can be between about 1.0 mm and about 3.0 mm. In these embodiments, the length of the major axis 302 can be between about 1.5 mm and about 3.5 mm.

In alternative embodiments, the length of the major axis 302 can be double the length of the minor axis 304.

FIG. 3A also illustrates that the opaque mask 102 can be shaped substantially as a circle (when the intraocular lens 100 is viewed in an en face or top-down direction). In these embodiments, the circular opaque mask 102 can surround or encircle the elliptical-shaped pinhole 300.

FIG. 3B illustrates an embodiment of an elliptical-shaped opaque mask 306 with an elliptical-shaped pinhole 300 such that the opaque mask 306 is substantially shaped as an elliptical ring. The elliptical-shaped opaque mask 306 can have a major axis and a minor axis. The length of the major axis of the elliptical-shaped opaque mask 306 can be greater than the length of the minor axis. As shown in FIG. 3B, the elliptical-shaped opaque mask 306 can completely surround the elliptical-shaped pinhole 300. The elliptical-shaped pinhole 300 can be a non-opaque or transparent portion of the optic portion 106 of the intraocular lens 100.

One technical problem faced by the applicant is how to design an extended depth of focus intraocular lens or how to increase the depth of focus of an implanted intraocular lens such that the lens is better suited to assist the subject in reading alphanumeric letters. One technical solution discovered and developed by the applicant is the intraocular lens 100 disclosed herein comprising an optic portion 106 having an opaque mask 102 formed within the optic portion 106 and enclosing an elliptical-shaped pinhole 104 or aperture. The applicant discovered that an intraocular lens 100 with an opaque mask 102 enclosing an elliptical-shaped pinhole 104 corresponds better to the average Fourier spectrum of certain alphanumeric letters. The applicant also discovered that intraocular lenses 100 with elliptical-shaped pinholes 104 can be more suited to those subjects that have a significant amount of “with the rule” astigmatism and can improve visual outcomes for those subjects.

FIG. 4 illustrates that the opaque mask 102 can be comprised of multiple mask layers 400. FIG. 4 is a schematic depiction of a cross-section of the optic portion 106 of the intraocular lens 100 when viewed from a side of the intraocular lens 100.

As shown in FIG. 4, the multiple mask layers 400 can comprise at least a first mask layer 400A and a second mask layer 400B. In some embodiments, the multiple mask layers 400 can further comprise a third mask layer 400C. Each of the mask layers 400 can be an opaque mask layer or a dark-colored mask layer (e.g., black-colored mask layer). At least one of the mask layers 400 (e.g., mask layer 400A) can be unaligned or staggered with respect to another one of the mask layers 400 (e.g., mask layer 400B) in an anteroposterior direction.

As previously discussed, the opaque mask 102 can be formed by directing laser light 402 or other forms of energy at layers of the optic portion 106 of the intraocular lens 100. In certain embodiments, the intraocular lens 100 can be implanted within an eye of a subject when the laser light 402 is directed at the optic portion 106 of the intraocular lens 100.

In some embodiments, the laser light 402 directed at the optic portion 106 can be generated by a femtosecond laser. For example, the laser light 402 can have a wavelength of between about 1030 nm and about 1064 nm (e.g., about 1053 nm).

In certain embodiments, the laser light 402 can have a pulse duration of between about 10,000 femtoseconds to about 10 femtoseconds and a pulse energy of between about 1 nanojoule (nJ) and 200 nJ.

As shown in FIG. 4, the first mask layer 400A and the second mask layer 400B (and, in some instances, the third mask layer 400C) can define a pinhole 104 or aperture. The pinhole 104 can be a non-opaque or transparent portion of the optic portion 106 surrounded by the opaque mask 102. The pinhole 104 can be made to be aligned or be centered with the visual axis 110 (see, e.g., FIG. 1) of an eye of a subject when the intraocular lens 100 is implanted within the eye of the subject. For example, as previously discussed in relation to FIG. 2B, the location of the visual axis 110 can be determined using the steps or operations discussed in operation 206.

In alternative embodiments, the location of the visual axis 110 can be estimated or determined using other ophthalmic methods or procedures.

As shown in FIG. 4, the opaque mask 102 can be comprised of a plurality of opaque spots 404 or opaque specks/stipples that are densely packed together or densely arranged within the optic portion 106.

Each of the opaque spots 404 can have a spot diameter or section diameter (when viewed in an en face or top-down direction). In some embodiments, the spot diameter or section diameter can be between about 2 μm and about 4 μm (or between about 3 μm and about 3.5 μm). The size of the spot diameter or section diameter can be configured or adjusted (increased or decreased) based on the size of the optic portion 106.

In certain embodiments, each of the opaque spots 404 or opaque specks/stipples can be spaced apart from another one of the opaque spots 404 or opaque specks/stipples (an immediately adjacent opaque spot 404 or opaque speck/stipple) in a radial or lateral direction. For example, each of the opaque spots 404 or opaque specks/stipples can be spaced apart from another one of the opaque spots 404 or opaque specks/stipples by between about 1 μm and about 5 μm (e.g., between about 2 μm and about 4 μm).

In some embodiments, each of the mask layers 400 can be spaced apart from one another (i.e., spaced apart from an immediately adjacent mask layer 400) in the anteroposterior direction. For example, each of the mask layers 400 can be spaced apart from one another in the anteroposterior direction by at least 10 μm. In other embodiments, each of the mask layers 400 can be spaced apart from one another in the anteroposterior direction by at least 5 μm or between about 10 μm and 30 μm.

FIG. 4 also illustrates that at least one of the mask layers 400 (e.g., mask layer 400A) can be arranged or positioned in a way that the mask layer 400 is unaligned or staggered with respect to another one of the mask layers 400 (e.g., mask layer 400B) in an anteroposterior direction. By arranging the mask layers 400 in this manner, the space (i.e., the non-opaque or transparent section) in between each of the opaque spots 404 or opaque specks/stipples is taken up or covered by another opaque spot 404 or opaque speck/stipple in a mask layer 400 below (or above) to make the entire mask appear opaque when viewed in the anteroposterior direction. Also, in this manner, the mask appears opaque or dark-colored (e.g., black-colored) while the pinhole 104 appears is left substantially transparent or non-opaque and the visual axis 110 of the eye of the subject is left unobstructed.

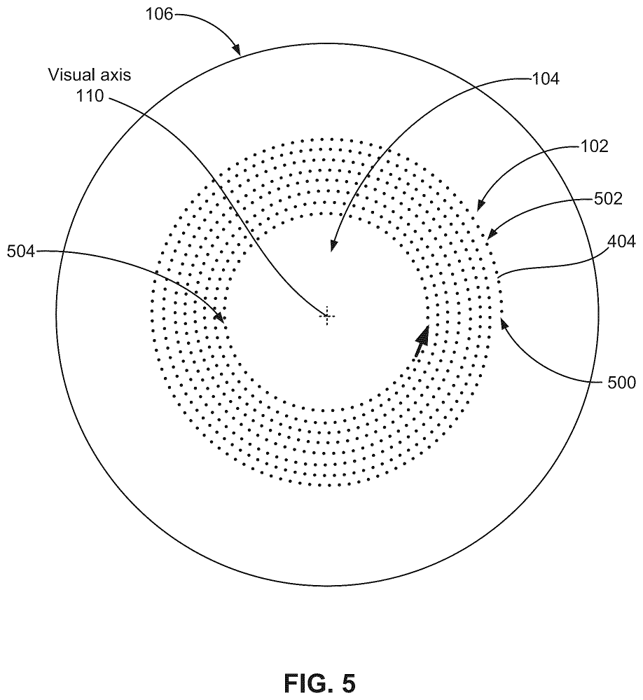

As shown in FIG. 4, one example method of forming a pinhole-apertured intraocular lens 100 can comprise directing laser light 402 at a first layer of the optic portion 106 of an intraocular lens in a first spiral pattern (see, e.g., FIG. 5) until a first opaque mask layer 400A is formed within the optic portion 106. The method can also comprise directing the laser light 402 at a second layer of the optic portion 106 of the intraocular lens in a second spiral pattern until a second opaque mask layer 400B is formed within the optic portion 106. The first opaque mask layer 400A can be unaligned or staggered with respect to the second opaque mask layer 400B in an anteroposterior direction.

In some embodiments, the second opaque mask layer 400B can be positioned or located anterior or posterior to the first opaque mask layer 400A.

The method can further comprise directing the laser light 402 at a third layer of the optic portion 106 of the intraocular lens in a third spiral pattern until a third opaque mask layer 400C is formed within the optic portion 106. The second opaque mask layer 400B can be unaligned or staggered with respect the third opaque mask layer 400C in an anteroposterior direction.

In some embodiments, the third opaque mask layer 400C can be positioned or located anterior or posterior to the second opaque mask layer 400B.

Although three opaque mask layers 400 are shown in FIG. 4, it is contemplated by this disclosure that the opaque mask 102 can comprise four, five, or more opaque mask layers 400.

In some embodiments, the intraocular lens can have a refractive power configured to adjust a refractive power of an eye of a subject to between about −0.7 diopters and about −0.9 diopters when the intraocular is implanted within the eye of the subject.

FIG. 5 illustrates that an annular-shaped opaque mask 102 can be created by directing laser light 402 (see, e.g., FIG. 4) at the optic portion 106 in a spiral pattern 500. As shown in FIG. 5, the spiral pattern 500 can begin at a radially outward portion of the optic portion 106 and can spiral radially inward such that a perimeter surrounding the pinhole 104 is formed last.

As previously discussed, the location of the visual axis 110 of an eye with an intraocular lens implanted therein can be determined first (e.g., using the steps of operation 206) and the desired location of the opaque mask 102 can be determined based on the location of the visual axis 110. For example, the location of the opaque mask 102 (i.e., where the laser light 402 will be directed) can be calculated or selected to ensure that the pinhole 104 of the opaque mask 102 is aligned or centered with the visual axis 110. As a more specific example, the location of the opaque mask 102 can be calculated or selected to ensure that the location of the pinhole 104 of the opaque mask 102 overlaps with or covers the location of the visual axis 110.

In alternative embodiments, the opaque mask 102 can be created by directing laser light 402 at the optic portion 106 in a starburst or radially outward pattern. In additional embodiments, the opaque mask 102 can be created by directing laser light 402 at the optic portion in an arch or curvilinear pattern.

As previously discussed, the opaque mask 102 (e.g., the annular-shaped opaque mask 102) can be comprised of a plurality of opaque spots 404 or opaque specks/stipples that are densely packed together or densely arranged within the optic portion 106. In some embodiments, the opaque mask 102 can be an apodized transmission mask such that a density of the opaque spots 404 or opaque specks/stipples is greater at a peripheral edge 502 of the opaque mask 102 than a portion of the opaque mask 102 radially inward of the peripheral edge 502 (e.g., along a middle portion or body of the annulus). In these and other embodiments, a density of the opaque spots 404 or opaque specks/stipples is greater also at an inner edge 504 of the opaque mask 102 than a portion of the opaque mask 102 radially outward of the inner edge 504 (e.g., along a middle portion or body of the annulus).

One technical problem faced by the applicant is how to increase the depth of focus of an intraocular lens without introducing visual disturbances or sacrificing visual acuity. One technical solution discovered and developed by the applicant is the intraocular lens 100 disclosed herein comprising an optic portion 106 having an opaque mask 102 formed within the optic portion 106 where the opaque mask 102 is an apodized transmission mask that can prevent visual disturbances caused by the peripheral edges of the opaque mask 102 being blurry or faded. The apodized transmission mask can enclose a pinhole 104 or aperture aligned or centered with a visual axis of the eye.

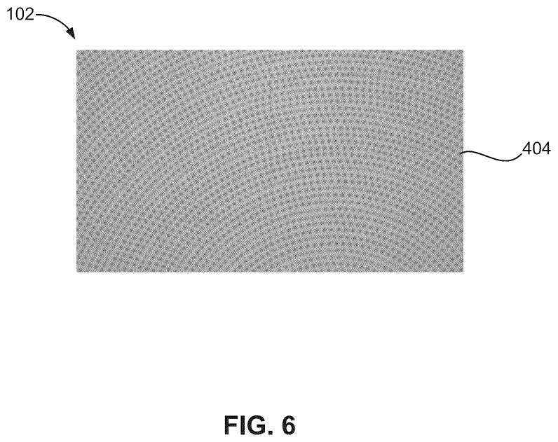

FIG. 6 is a close-up image of a plurality of opaque spots 404 or opaque specks/stipples making up part of an opaque mask 102. As shown in FIG. 6, the opaque spots 404 or opaque specks/stipples are arranged in spiral pattern. Each of the opaque spots 404 or opaque specks/stipples can be spaced apart from another one of the opaque spots 404 or opaque specks/stipples.

FIG. 6 illustrates only one mask layer of the opaque mask 102. The mask layer can be created by directing laser light (e.g., from a femtosecond laser) at the optic portion 106 of the intraocular lens 100 in a spiral pattern. Additional mask layers can be created anterior or posterior of the mask layer shown in FIG. 6. The mask layers can also be separated or spaced apart from one another in an anteroposterior direction by at least 5 μm or between about 10 μm and 30 μm. Each of the mask layers can be unaligned or staggered with respect to another one of the mask layers in an anteroposterior direction.

FIG. 7 is an image of one embodiment of an intraocular lens 100 comprising a substantially annular-shaped opaque mask 102 defining a pinhole 104. As shown in FIG. 7, the opaque mask 102 can be substantially black-colored. The black color of the opaque mask 102 can be a result of the laser light (e.g., laser light produced by the femtosecond laser) carbonizing the biocompatible polymeric material making up the optic portion 106 of the intraocular lens 100.

As previously discussed, the optic portion 106 can be made of a biocompatible polymeric material such as a transparent hydrophobic acrylic material. Directing the laser light at the optic portion 106 can result in the laser light heating up portions of the biocompatible material (e.g., acrylic material) until the portions of the biocompatible material turns opaque or undergoes a carbonization process such that the once transparent material is now opaque or black-colored.

FIG. 7 illustrates that the location of the pinhole 104 (the transparent portion of the optic portion 106) need not be concentric with a peripheral edge of the optic portion 106 and that the location or placement of the pinhole 104 (as well as the location or placement of the opaque mask 102) is dictated by the location of the visual axis.

One technical problem faced by the applicant is how to adjust a depth of focus of an implanted intraocular lens or an intraocular lens that was not initially made to be an extended depth of focus intraocular lens. One technical solution discovered and developed by the applicant is the method disclosed herein comprising determining a location of a visual axis of an eye comprising an IOL implanted therein, adjusting a refractive power of the eye with the IOL implanted therein to between about −0.7 diopters and about −0.9 diopters, and directing laser light at an optic portion in a spiral pattern until an opaque mask is formed within part of the optic portion enclosing a pinhole aligned with the visual axis. The method can produce an intraocular lens 100 with an optic portion 106 having an opaque mask 102 formed within the optic portion 106 and enclosing a pinhole 104 or aperture. The location or placement of the pinhole 104 can be aligned or centered with a visual axis of the eye.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having,” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about,” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A method of increasing a depth of focus of an intraocular lens (IOL), comprising:
determining a location of a visual axis of an eye comprising an IOL implanted therein;
adjusting a refractive power of the eye with the IOL implanted therein to between about −0.7 diopters and about −0.9 diopters; and
directing laser light at an optic portion of the IOL in a spiral pattern until an opaque mask is formed within part of the optic portion enclosing a pinhole aligned with the visual axis.

2. The method of claim 1, wherein the pinhole is shaped substantially as a circle.

3. The method of claim 1, wherein the pinhole is shaped substantially as an ellipse.

4. The method of claim 1, wherein the pinhole is a non-opaque portion of the optic portion surrounded by the opaque mask.

5. The method of claim 1, wherein the pinhole has a pinhole diameter, wherein the pinhole diameter is between about 1.0 mm and about 3.0 mm.

6. The method of claim 5, wherein the pinhole diameter is between about 1.8 mm and about 2.2 mm.

7. The method of claim 1, wherein the opaque mask has a mask outer diameter or mask width, and wherein the mask outer diameter or the mask width is between about 3.0 mm and about 5.0 mm.

8. The method of claim 1, wherein the laser light is generated by a femtosecond laser.

9. The method of claim 8, wherein the laser light has a wavelength of between about 1030 nm and about 1064 nm.

10. The method of claim 1, wherein the opaque mask is a black-colored mask.

11. The method of claim 1, wherein the opaque mask is shaped substantially as an annulus.

12. The method of claim 1, wherein the opaque mask is shaped substantially as an elliptical ring.

13. The method of claim 1, wherein the opaque mask is comprised of a plurality of opaque spots densely packed together within the optic portion.

14. The method of claim 13, wherein each of the opaque spots has a spot diameter, and wherein the spot diameter is between 3 μm and 4 μm.

15. The method of claim 13, wherein the opaque mask is an apodized transmission mask such that a density of the opaque spots is greater at a peripheral edge of the opaque mask than a portion of the opaque mask radially inward of the peripheral edge.

16. The method of claim 1, further comprising:
implanting the IOL within the eye of a subject; and
waiting for at least 90 days after the IOL is implanted before determining the location of the visual axis.

17. The method of claim 1, wherein the pinhole is aligned with the visual axis when at least part of the pinhole overlaps with the visual axis.

18. The method of claim 1, wherein the opaque mask comprises multiple mask layers, wherein at least one of the mask layers is not aligned with another one of the mask layers in an anteroposterior direction.

19. (canceled)

20. (canceled)

21. A method of forming a pinhole intraocular lens (IOL), comprising:
directing laser light at a first layer of an optic portion of an IOL in a first spiral pattern until a first opaque mask layer is formed within the optic portion, wherein the IOL has a refractive power configured to adjust a refractive power of an eye to between about −0.7 diopters and about −0.9 diopters when the IOL is implanted within the eye; and
directing the laser light at a second layer of the optic portion of the IOL in a second spiral pattern until a second opaque mask layer is formed within the optic portion,
wherein the first opaque mask layer is not aligned with the second opaque mask layer in an anteroposterior direction.

22.-40. (canceled)

41. An intraocular lens (IOL), comprising:
an optic portion and one or more haptics coupled to the optic portion; and
an opaque mask formed within the optic portion,
wherein the opaque mask encloses a pinhole,
wherein the opaque mask is comprised of multiple mask layers, and
wherein at least one of the multiple mask layers is not aligned with another one of the multiple mask layers in an anteroposterior direction.

42.-60. (canceled)
